(19) United States
(12) Patent Application Publication
(10) Pub. No.: US 2019/0194652 A1
(43) Pub. Date: Jun. 27, 2019

(54) HIV CLINICAL PLAN

(71) Applicant: EXCISION BIOTHERAPEUTICS. INC., Andover, NJ (US)

(72) Inventor: Thomas MALCOLM, Andover, NJ (US)

(73) Assignee: EXCISION BIOTHERAPEUTICS, INC., Andover, NJ (US)

(21) Appl. No.: 16/290,343

(22) Filed: Mar. 1, 2019

Related U.S. Application Data
(63) Continuation-in-part of application No. PCT/US17/51093, filed on Sep. 12, 2017.

(51) Int. Cl. C12N 15/11; C12N 9/22; A61K 9/00; C12Q 1/70; G01N 33/569

(52) U.S. Cl. CPC C12N 15/11; C12N 9/22; A61K 9/0019; C12Q 1/70; G01N 2800/52; C12N 2310/20; C12N 2800/80; C12N 2320/35; G01N 2333/16; G01N 33/56988

(57) ABSTRACT

A method of performing a clinical trial for a gene editing or gene excising system for treating HIV in humans, by recruiting HIV infected individuals currently receiving highly active antiretroviral therapy (HAART) that is effective in lowering viral load and entering qualified individuals as participants in a clinical trial, administering the gene editing or gene excising system treatment to the participants in Phase 1a, Phase 1b, and Phase 1c, and performing assays to confirm HIV viral genome excision from the participants' cells. A method of performing a clinical trial for a gene editing or gene excising system for treating a latent viral infection in humans.

### BACKGROUND OF THE INVENTION

### 1. Technical Field

The present invention relates to methods of performing clinical trials with the purpose of determining safety and tolerability while obtaining confirmation of mechanism-of-action and further obtaining dosing information to guide the design of subsequent clinical trials. More specifically, the present invention relates to methods of performing clinical trials with gene therapeutics.

### 2. Background Art

Clinical trials with human participants are required by the Federal Drug Administration (FDA) in order for it to approve the safety and effectiveness of a medical treatment. Clinical trials are required for all new drugs, biologics, gene therapies, dietary supplements, and medical devices. Generally, a small pilot study is performed first and subsequently larger studies are performed. The human participants usually are suffering from some medical condition that the new treatment is designed to remedy. If it is found that the benefits of the new treatment outweigh the risks, the FDA will approve the new treatment for its intended use.

To date, no gene therapeutics have been approved by the FDA, although many are being studied in clinical trials. The first clinical trial for a CRISPR-Cas9 system (clustered regularly interspaced short palindromic repeats) has just recently been approved to begin in order to determine whether CRISPR is safe to use in humans. CRISPR-Cas9 is a gene editing system derived from microbial organisms that can be used to insert, delete, or otherwise mutate an organism's genome by the use of nucleases. Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity permitting the cutting of target DNA. Cas9 is guided by a mature crRNA that contains about 20 base pairs (bp) of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or can be engineered into an artificial fusion small guide RNA (sgRNA) via a synthetic stem loop (AGAAAU) in order to mimic the natural crRNA/tracrRNA duplex. Such sgRNA, like shRNA, can be synthesized or can be transcribed in vitro for direct RNA transfection or can be expressed from a U6 or H1-promoted RNA expression vector, although cleavage efficiencies of the artificial sgRNA are lower than those for systems with the crRNA and tracrRNA expressed separately. Other companies pursuing CRISPR and CRISPR-like technologies, including CRISPR Inc. (Basel, Switzerland), Editas (Cambridge Mass.), and Caribou (Berkeley Calif.) are utilizing these technologies to create specific gene edits or block gene expression as opposed to deleting large segments of a viral genome.

CRISPR can be used in treating many different viruses by inactivating the viruses or by deleting the viral genome from the host's DNA. For example, U.S. patent application Ser. No. 14/838,057 to Khalili, et al. discloses a method of inactivating a proviral DNA integrated into the genome of a host cell latently infected with a retrovirus, including the steps of: treating the host cell with a composition comprising a Clustered Regularly Interspaced Short Palindromic Repeat (CRISPR)-associated endonuclease, and two or more different guide RNAs (gRNAs), wherein each of the at least two gRNAs is complementary to a different target nucleic acid sequence in a long terminal repeat (LTR) of the proviral DNA; and inactivating the proviral DNA. Preferably, the proviral DNA being inactivated is human immunodeficiency virus (HIV).

HIV/AIDS remains a major public health issue in much of the world, despite the increased availability of antiretroviral therapy (ART) that effectively suppresses viral replication as measured in the plasma using a PCR based assay. The development of suppressive combinations of ART in the 1990s has reduced substantially the morbidity and mortality from HIV-1 infection in those patients who adhere well to the ART regimen. In these patients, HIV-1 infection has become a chronic condition where progression to AIDS is rare. However, chronic ART is not without clinically significant adverse effects, including cardiovascular disease, non-AIDS malignancies, and osteoporosis. For years, substantial research effort has been directed at developing ART regimens that are more effective (suppressive), easier to adhere to (e.g., once daily dosing) and/or have fewer adverse effects.

Even in the presence of suppressive ART with undectable viral load in the plasma, the virus persists in the body in the form of integrated proviral DNA in latently infected cells, especially in lymphocytes and other monocytes. Discontinuation of ART almost always leads to the re-emergence of detectable viral replication, rebound in viral load, and the progression of immunological and clinical manifestations of HIV infection. Therefore, a safe and effective strategy to eradicate HIV reservoirs is needed to effect a functional cure without the need for chronic ART.

The properties of the latently infected cells that constitute the HIV reservoirs make viral eradication a formidable challenge. Latent cellular reservoirs exist in sites throughout the body, including in circulating blood and in the central nervous system (CNS), bone marrow, and gut-associated lymphoid tissue (GALT). It has been estimated that latently infected CD4+ T cells can live for many decades, are resistant to ART, and are not susceptible to attack by the immune system.

There remains a need for a clinical trial design for CRISPR gene editing systems, especially as a treatment for HIV.

### SUMMARY OF THE INVENTION

The present invention provides for a method of performing a clinical trial for a gene editing or gene excising system for treating HIV in humans, by recruiting HIV infected individuals currently receiving highly active antiretroviral therapy (HAART) that is effective in lowering viral load and entering qualified individuals as participants in a clinical trial, administering the gene editing or gene excising system treatment to the participants in Phase 1a, Phase 1b, and Phase 1c, and performing assays to confirm HIV viral genome excision from the participants' cells.

The present invention also provides for a method of performing a clinical trial for a gene editing or gene excising system for treating a latent viral infection in humans, by recruiting infected individuals and entering qualified individuals as participants in a clinical trial, administering the gene editing or gene excising system treatment to the participants in Phase 1a, Phase 1b, and Phase 1c, and performing assays to confirm viral genome excision from the participants' cells.

### DESCRIPTION OF THE DRAWINGS

Other advantages of the present invention are readily appreciated as the same becomes better understood by reference to the following detailed description when considered in connection with the accompanying drawings wherein:

FIG. 1 is a schematic of the trial design of the method; and

FIG. 2 is a table of study procedures occurring during different days and weeks of the method.

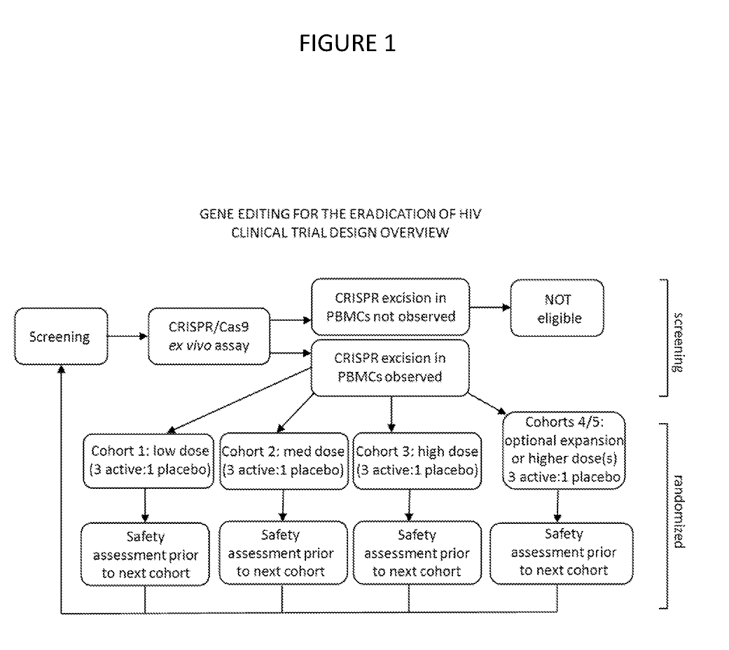

### DETAILED DESCRIPTION OF THE INVENTION

The present invention provides for methods of performing a clinical trial for a gene editing or gene excising system to treat latent viral infections, especially HIV, in humans. Most generally, the method includes performing a clinical trial for a gene editing or gene excising system for treating a latent viral infection in humans, by recruiting infected individuals and entering qualified individuals as participants in a clinical trial, administering the gene editing or gene excising system treatment to the participants in Phase 1a, Phase 1b, and Phase 1c, and performing assays to confirm viral genome excision from the participants' cells. Specifically for HIV, the method includes recruiting HIV infected individuals currently receiving and responding well to highly active antiretroviral therapy (HAART) (i.e. it is effective in lowering viral load), administering the gene editing or gene excising system treatment to the individuals in Phase 1a, Phase 1b, and Phase 1c, and performing assays to confirm HIV viral genome excision from the individuals' cells.

The clinical trial can be used for assessing the safety and tolerability of a single ascending dose of the gene editing system treatment. The clinical trial can also be used for assessing the pharmacodynamic (PD) (in vivo HIV proviral DNA gene-editing activity) of the gene editing system treatment in PBMCs and in lymph tissue from rectal biopsies, for assessing the immunogenicity of the gene-editing system treatment by measuring anti-AAV9 serum antibodies, and for assessing the PK (distribution and persistence) of the gene editing system treatment using a vector specific DNA qPCR assay in blood and in lymph tissue from rectal biopsies, as available. The clinical trial can also be used for analyzing the effect of the gene editing system treatment on a viral outgrowth assay in PBMCs and its correlation with gene-editing activity.

The gene editing or gene excising system is preferably, but not limited to, a CRISPR system or Argonaute system and effectively excises the entire genome of a virus such as HIV from the host cells. Most preferably, the treatment is EBT101 (CRISPR-Cas9 system (AAV9-SaCas9.U6sgLTR-1.U6sgGagD) using at least two gRNAs targeting the U3 region of the 3′ and 5′ LTR (long terminal repeat sequence), and the Gag and Pol genes of the HIV-1 pro and non-integrated virus).

The active biological agent, AAV9-SaCas9.U6sgLTR-1.U6sgGagD, is formulated in phosphate buffered saline containing 5% sorbitol and other compendial excipients. EBT-101 is formulated at a concentration of approximately 1×10³ vg/mL in phosphate buffered saline containing 180 mM sodium chloride, 10 mM sodium phosphate, and 0.001% Poloxamer 188 (pH 7.3). Vials contain either 1 mL of formulated EBT-101 or placebo and are labeled for Investigational Use Only. Dosing vials are preferably stored at −80° C.

Preferably, the gene editing or gene excising system is administered intravenously (IV) by infusion at a rate of ≤25 mL/hr at a volume of 100 mL using an automatic volumetric infusion pump. Dosing vials can be thawed two hours prior to administration.

Three dose levels of EBT101 are preferably used in the method: a low dose of 1.0×10¹² vg/kg, a middle dose of 3×10¹² vg/kg, and a high dose of 1.0×10¹³ vg/kg. The starting dose in humans will approximate the low dose in a SIV monkey study and the highest dose will not exceed the top dose in the SIV monkey study.

“Nuclease” as used herein, refers to an enzyme that is able to cleave the phosphodiester bonds between nucleotide subunits of nucleic acids.

The CRISPR system can use a Cas nuclease (such as Cas9) or a Cpf1 nuclease, or any other suitable nuclease that is able to target DNA or RNA and make additions, deletions, mutations, and preferably excisions of entire genes or gene clusters.

The Argonaute system is an RNA-guided or DNA-guided endonuclease enzyme that is able to cleave any sequence complementary to guide RNA or guide DNA. Argonaute proteins are proteins of the PIWI protein superfamily that contain a PIWI (P element-induced wimpy testis) domain, a MID (middle) domain, a PAZ (Piwi-Argonaute-Zwille) domain and an N-terminal domain. Argonaute proteins are capable of binding small RNAs, such as microRNAs, small interfering RNAs (siRNAs), and Piwi-interacting RNAs. Argonaute proteins can be guided to target sequences with these RNAs in order to cleave mRNA, inhibit translation, or induce mRNA degradation in the target sequence. Natronobacterium gregoryi Argonaute (NgAgo) is a DNA-guided endonuclease suitable for genome editing in human cells.

“Placebo” as used herein, refers to a substance with no pharmaceutical effect. The placebo used is preferably phosphate buffered saline containing 5% sorbitol and other compendial excipients administered by IV infusion at a rate of ≤25 mL/hr using an automatic volumetric infusion pump.

“Dose limiting toxicity” (DLT) as used herein, refers to any one or more of the following: a Grade 3 AE (Common Technical Criteria for Adverse Events [CTCAE]) that is likely or definitely related to EBT-101 per the investigator, any Grade 4 AE, a 30% decrease of the CD4 T cell count persisting for ≥4 weeks, or rebound of plasma HIV-1 RNA >1,000 copies/ml, unrelated to poor adherence to ART and confirmed by a second measurement within one week.

“Maximum tolerated dose” (MTD) as used herein, refers to the dose below which a DLT occurred and at which 8 participants (including 2 placebo) tolerated the dose without DLT.

“Adverse event” as used herein, refers to any untoward medical occurrence associated with the use of a drug in humans, whether or not considered drug related.

“Serious adverse event” as used herein, refers to an adverse event that results in any of the following outcomes: death, life-threatening (in the view of either the Investigator or Sponsor, its occurrence places the participant at immediate risk of death, it does not include an AE that, had it occurred in a more severe form, might have caused death), inpatient hospitalization or prolongation of existing hospitalization, a persistent or significant incapacity or substantial disruption of the ability to conduct normal life functions, a congenital anomaly/birth defect, or important medical events that may not result in death, be life-threatening, or require hospitalization can be considered serious when, based upon appropriate medical judgment, they may jeopardize the participant and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

The latent viral infection being treated in the clinical trial can be caused by either a lytic or lysogenic virus. Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis) and the virions go on to infect additional cells. Lytic viruses can integrate viral DNA into the host genome as well as be non-integrated where lysis does not occur over the period of the infection of the cell.

“Lysogenic virus” as used herein, refers to a virus that replicates by the lysogenic cycle (i.e. does not cause the host cell to burst and integrates viral nucleic acid into the host cell DNA). The lysogenic virus can mainly replicate by the lysogenic cycle but sometimes replicate by the lytic cycle. In the lysogenic cycle, virion DNA is integrated into the host cell, and when the host cell reproduces, the virion DNA is copied into the resulting cells from cell division. In the lysogenic cycle, the host cell does not burst. Lysogenic viruses treated with the compositions and methods of the present invention can include, but are not limited to, hepatitis A, hepatitis B, hepatitis D, HSV-1, HSV-2, cytomegalovirus, Epstein-Barr virus, Varicella Zoster virus, HIV1, HIV2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, and BK virus.

“Lytic virus” as used herein refers to a virus that replicates by the lytic cycle (i.e. causes the host cell to burst after an accumulation of virus within the cell). The lytic virus can mainly replicate by the lytic cycle but sometimes replicate by the lysogenic cycle. Lytic viruses treated by the compositions and methods of the present invention can include, but are not limited to, hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

The method provides for a randomized, placebo controlled, single ascending dose, sequential cohort Phase 1 trial of EBT-101 administered intravenously to aviremic HIV-1 infected adults on stable ART. The duration of the main study is 24 weeks for each participant. There are three dose levels, one per cohort, each cohort including four participants randomized 3:1 to active drug or to matching placebo. The 4th cohort and potentially a 5th cohort can receive the maximum tolerated dose or the 3rd dose level (to expand this cohort to 8 participants) or a higher dose (if safety and PD data support a higher dose), per the recommendations of the independent Data Monitoring Committee (DMC) and concurrence of the Sponsor. The total duration of the main study for all participants is a minimum of 80 weeks based on minimum intervals between dosing of participants within and between a maximum of 5 cohorts. Participants can have clinic visits scheduled for safety endpoints (AEs and safety labs) at the baseline visit and on Days 1, 2, and 3 post dose; and at 1, 2, 4, 8, 16, and 24 weeks post dose. Blood samples can be obtained at baseline and at 1, 2, 4, 8, 16, and 24 weeks post dose, for PD (gene editing activity and viral outgrowth) as needed. Each participant's ART regimen will be continued throughout the study unless viral rebound indicates a need for change(s) in drugs or doses. Changes to the ART regimen should be made only after consultation with the study medical monitor.

The screening period can be 3-12 weeks in duration since a minimum of 2 weeks is required for the results of an ex vivo gene editing assay to become available. The maximum period of 12 weeks allows for this assay to be repeated, if necessary. If the screening period (starting with signing of informed consent form) extends beyond 12 weeks, a repeat assessment of other eligibility criteria must be conducted for eligibility, except for the gene editing assay, which does not need to be repeated.

Eligible participants can consent to undergo an optional rectal biopsy for lymph tissue at baseline and at 4 weeks, which will be tested for gene editing activity. Participants will be monitored for AEs, safety laboratory values, plasma viral load, peripheral blood CD4 counts, and other PD assays for 24 weeks post dose. Additional blood samples can be obtained and stored for future assays at baseline, 4 weeks, and 24 weeks.

All participants will be enrolled in a Long Term Follow-up (LTFU) protocol after completing the 24 week main study. In this protocol, there are annual clinic visits for 5 years post dose followed by annual telephone contact for an additional 10 years (15 years total post dose monitoring) to assess adverse events. During the LTFU study, participants are monitored for any new onset of medical conditions including malignancies or changes in existing medical conditions. Participants can be asked to give consent for health records from their health care providers to be sent directly to the investigator during this follow-up period.

The method extends through the end of Phase 1 to establish safety, tolerability, and effective excision/deletion Mechanism-of-Action by way of existing standard PCR assays and ELISA assays used in current clinical studies. A biochemical metabolomics based Mass Spectrometry/NMR diagnostics assay can be used in parallel with the standard PCR assays and ELISA assays to validate the metabolomics assays increased sensitivity, specificity, selectivity, robustness and precision for future use as an independent companion diagnostic. This assay is further described below.

Before or during the recruitment step, or during the trial itself, toxicology of EBT-101 can be assessed in two animal models: HIV Tg26 transgenic mouse model, and simian immunodeficiency virus (SIV)-infected rhesus macque model. In the mouse study, male mice (10/group) are assigned to one of four dose levels or control vehicle. EBT-101 can be delivered by IV injection via the tail vein. Mice can be euthanized and samples can be collected at 7 and 90 days post-injection. The study assesses the excision efficacy and safety at doses of 4×10¹¹ to 1.2×10¹⁴ vg/kg.

In SIV-infected rhesus macaque monkeys, an SIV homologue of EBT-101 can be used to assess excision efficacy and safety of AAV9-SaCas9.SIVmac239.U6sgLTR.U6sgGag. This SIV model can be conducted in monkeys with SIV under ART therapy to parallel the Phase 1 clinical trial with EBT-101 in aviremic HIV-Infected adults receiving ART. Single doses of 1.4×10¹² vg/kg and 1.4×10¹³ vg/kg will be tested.

For the recruitment step, these trials can recruit individuals having latent viral infections. More specifically, the trial can recruit HIV infected individuals (males and females) who are responding well to HAART (highly active antiretroviral therapy) (i.e. the HAART treatments are effective) as measured by (1) no viral replication, (2) no viral load (undetectable, ≤20 copies/mm3), and (3) healthy CD4 T cell counts (>500/mm3). The HAART treatments which the individuals are currently taking can be nucleoside reverse transcriptase inhibitors, non-nucleoside reverse transcriptase inhibitors, protease inhibitors, fusion inhibitors, integrase inhibitors, CCR5 antagonists, or combinations thereof. This study population was chosen because of risk/benefit considerations. Relatively healthy patients with few co-morbidities should tolerate any potential adverse effects better than a more advanced HIV-1 infected population. Also, it should be easier to interpret the clinical significance and potential drug relatedness of any adverse events in an otherwise largely asymptomatic population. Finally, since EBT-101 is not expected to result in gene editing in cells with actively replicating HIV-1, it is important to study an aviremic population.

Approximately 60 participants can be screened to enroll and dose up to 20 participants. Any participant who withdraws consent prior to 4 weeks post dose will be replaced.

During recruitment, potential participants are screened to assess eligibility for the study within 3-12 weeks prior to Study Day 1 (Randomization). Blood for the ex vivo CRISPR/Cas9 gene editing assay is obtained after the potential participant has met all other eligibility criteria. This assay may be repeated if necessary. If the screening period (starting with signing of informed consent form) extends beyond 12 weeks, a repeat assessment of other eligibility criteria must be conducted except for the gene editing assay, which does not need to be repeated.

The following assessments and procedures are performed and data collected during the screening period to aid in evaluating inclusion and exclusion criteria described below. These assessments can also be performed during the study itself.

1. Written informed consent for main and LTFU protocols.

2. Demographics (Age, gender, race and ethnicity).

3. Height, weight (Body weight can be measured in kilograms. Height can be measured in centimeters).

4. Medical history (A complete medical history detailing other co-morbid conditions and history of complications of HIV can be recorded).

5. Full physical examination (including head, ears, eyes, nose and throat (HEENT), neurological, musculoskeletal, chest, lungs, abdomen, cardiovascular, genitourinary and rectal). A targeted physical examination can be performed as indicated based on sign, symptoms, and/or adverse events.

6. Vital signs (Temperature, heart rate, blood pressure. Heart rate and blood pressure should be obtained after participant is seated for ≥5 minutes).

7. Laboratory safety tests (Serum chemistries: sodium, potassium, chloride, bicarbonate, blood urea nitrogen (BUN), calcium, creatinine, glucose, albumin, phosphorus, alanine aminotransferase (ALT), aspartate aminotransferase (AST), alkaline phosphatase (ALP); Hematology: red blood cell (RBC), hemoglobin, hematocrit, platelet count, white blood cell (WBC), differential (neutrophils, lymphocytes, monocytes, eosinophils, basophils).

8. CD4 T cell count (Peripheral blood sample to monitor immunologic status).

9. HIV-1 RNA (plasma PCR assay).

10. AAV9 antibodies (serum).

11. Serum pregnancy test for women of child bearing potential (not surgically sterile or post-menopausal defined as age >40 years without menses for 22 years).

12. Urinalysis (Appearance, bilirubin, blood, color, glucose, ketones, leukocyte esterase, nitrates, pH, protein, specific gravity, and urobilinogen. Microscopic urinalysis will be performed at screening and at other visits only if urine dipstick results are abnormal).

13. 12-Lead electrocardiogram (ECG).

14. Ex vivo CRISPR/Cas9 gene editing assay, pharmacokinetic assessments (a PCR-based assay that will be performed in PBMCs and in rectal biopsy samples (obtained only after the potential participant has met all other eligibility criteria, and may be repeated if necessary)

15. P53 Mutation assay (XXX based assay that can be performed in PBMCs to monitor for any changes that potentially could be oncogenic).

16. Pharmacokinectic assessment (Blood sample for vector specific DNA qPCR detection assay).

17. Viral outgrowth assay (An ex vivo viral outgrowth assay can be performed periodically in cultured PBMCs from patients following study drug administration to investigate the potential for HIV-1 replication in the absence of ART).

18. Concomitant medications for 3 months prior to screening.

19. Blood samples can generally be collected for any additional assays.

In general, the participants must meet the following inclusion criteria to be included in the trials. Any appropriate assays or tests can be performed or documentation collected to obtain the necessary information as above.

1. Willing and able to provide written, signed informed consent prior to initiation of any study related procedures.

2. Age ≥18 years.

3. Documented HIV-1 infection.

4. Antiretroviral therapy (ART) for >1 year prior to screening

5. Plasma HIV-1 RNA <200 copies/mL for 21 year prior to screening (based on at ≥3 measurements during the year prior to screening).

6. Stable ART (no changes in drugs or doses) for >3 months prior to screening and expectation that current ART regimen will be maintained throughout study duration.

7. Plasma HIV-1 RNA <20 copies/mL (minimum of two assay results at least one week apart) within 30 days prior to screening and/or during screening.

8. Peripheral blood CD4 T cell count >500 cells/mm3 (minimum of two assay results at least one week apart) within 30 days prior to screening and during screening.

9. Hemoglobin: ≥10.0 (males); ≥9.0 (females) g/dL.

10. Absolute neutrophil count (ANC): ≥1000/mm3.

11. Platelet count: ≥75,000/mm3.

12. Serum creatinine: 1.5 mg/dL (133 μmol/L).

13. Aspartate aminotransferase (AST) or alanine aminotransferase (ALT): ≤2.5 times the upper limit of normal (ULN).

14. In blood sample from patient, ex vivo gene editing activity of EBT-101 demonstrated in at least 20% of transduced cells.

15. Willing and able to comply, as assessed by the investigator, with all study related procedures.

16. Willing and able to consent to enrollment in the LTFU protocol (15 years) after completing the main study.

17. Females of child-bearing potential must have a negative serum pregnancy test at screening and negative urine test prior to dosing.

18. Sexually active females of child-bearing potential must be willing to use one or more of the following forms of contraception from the time of providing written informed consent through the 24 week post dose visit: hormonal (i.e., oral, transdermal, implant, or injection); barrier (i.e., condom, diaphragm with spermicide); intrauterine device (IUD); vasectomized partner (6 months minimum); or abstinence. (Females who have been in menopause for at least 2 years, have had a tubal ligation at least 1 year prior to screening, or have had a total hysterectomy are not required to use any other forms of contraception during the study).

19. Sexually active males must be willing to use one or more of the above forms of birth control for either themselves or their partner, as appropriate, from the time of signing the informed consent form throughout the 24 week post dose visit. (Males who are post-vasectomy for at least 2 years with no known pregnancies in sexual partners are not required to use any other forms of contraception during the study).

The recruitment step can also use the following exclusion criteria such that any individuals who meet any one or more of the exclusion criteria are deemed not eligible to participate in the study. Any appropriate assays or tests can be performed or documentation collected to obtain the necessary information.

1. Use of any investigational product or investigational medical device within 30 days prior to screening, or anticipated use of any investigational agent prior to completion of all scheduled study visits through one year post dose.

2. Known or suspected hypersensitivity to any components of EBT-101 including formulation excipients and AAV9.

3. Pregnant or breastfeeding or planning to become pregnant (self or partner) or to breastfeed at any time through 24 weeks post dose.

4. Concurrent disease or condition (e.g. history or presence of clinically significant cardiovascular, pulmonary, hepatic, renal, hematologic, gastrointestinal, endocrine, immunologic, dermatologic, neurological, oncologic, or psychiatric disease) that, in the opinion of the investigator, would interfere with study participation or safety.

5. Major surgery planned during the 24 week study period.

6. History of malignancy with the exception of successfully treated basal cell or squamous cell carcinoma of the skin.

7. History of HIV-related opportunistic infections within the past 5 years.

8. Receipt of any experimental HIV vaccine (prophylactic and/or therapeutic) within the past year.

9. Receipt of any gene therapy product, approved or experimental, at any time.

10. Receipt of any licensed or experimental non-HIV vaccine (e.g., hepatitis B, influenza, pneumococcal polysaccharide) within the 30 days prior to screening.

11. Acute or serious illness, in the opinion of the site investigator, requiring systemic treatment and/or hospitalization, within 30 days prior to screening.

12. A history of organ transplantation or of chronic immunosuppressive therapy.

13. Receipt of any systemic immunomodulator (e.g., corticosteroids, interleukins, interferons) within 60 days prior to screening or anticipated use during the 24 week study period.

14. Immune deficiency other than from HIV-1 infection.

15. Active drug or alcohol use or dependence that, in the opinion of the site investigator, would interfere with adherence to study requirements.

16. The presence of detectable anti-AAV9 antibodies.

Upon determination that a participant meets all eligibility criteria, they are enrolled into the study and randomized 3:1 to active study drug or to placebo in the open cohort. Cohorts of 4 participants each will receive one of three successively higher dose levels of EBT-101 or matching placebo, with two additional cohorts possibly receiving higher dose(s) depending on safety and PD at the lower doses. Participants in each cohort will be randomized 3:1 to active drug or to placebo.

Participants within each cohort will be dosed in a staggered fashion no closer than 2 weeks apart. The first participant in a new cohort will be dosed no sooner than 4 weeks after dosing of the last participant in the previous cohort. Participants in cohorts who are receiving the same or a lower dose than the highest dose already tested may be enrolled with a minimum of one week between enrollment of each successive participant.

An independent Data Monitoring Committee (DMC) will review all available data after the last participant in each cohort completes 2 weeks of post dose monitoring. If this review indicates no DLT or other safety concerns, the next eligible participant will be enrolled into the next higher dose cohort a minimum of 4 weeks following dosing of the last participant in the prior cohort. A minimum of 2 weeks between enrollment of each successive participant within a cohort will continue. If a DLT is observed in any participant, the DMC may recommend to the sponsor: request additional safety data, halt further enrollment of participants, add a cohort at the previous lower dose, dose a cohort with an intermediate lower dose, stop the study, continue enrolling additional participants within the cohort, and/or proceed with dose escalation in the next cohort.

Before the study starts, investigators, participants, and all study staff with direct contact with participants can be blinded to treatment assignment. A designated unblinded pharmacist at each site can prepare each dose. That individual should have no contact with the participants and minimize contact with other site study personnel.

Unblinding of treatment assignment is discouraged. In the event of a medical emergency, where the identity of the treatment assignment is critical to the care of a participant, the Investigator should call the Medical Monitor to discuss. In the event that unblinding is deemed necessary, a designated unblinded study team member will provide the treatment assignment to the Medical Monitor who will provide the information to the Investigator. Treatment assignment may also be unblinded by the Medical Monitor in the event a stopping criterion is met per DLT.

Individuals will not be weaned from anti-viral (HAART) therapy that they are currently taking and can remain on the HAART throughout the clinical trial. Individuals will not change drugs or doses unless clinically indicated after consultation with the study medical monitor. All concomitant medications, including ART, will be recorded during screening and during the 24 week period of the main study.

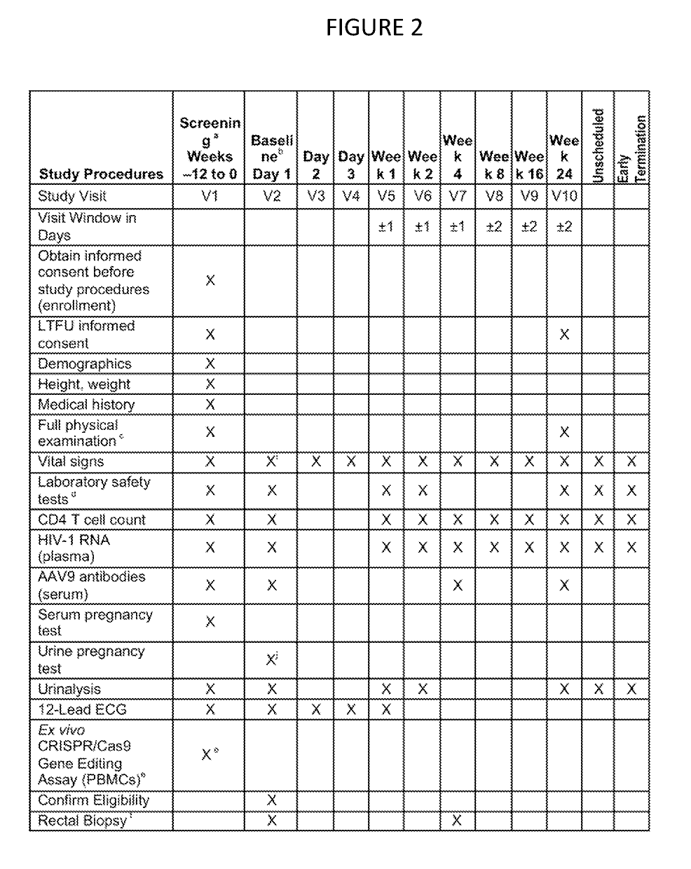

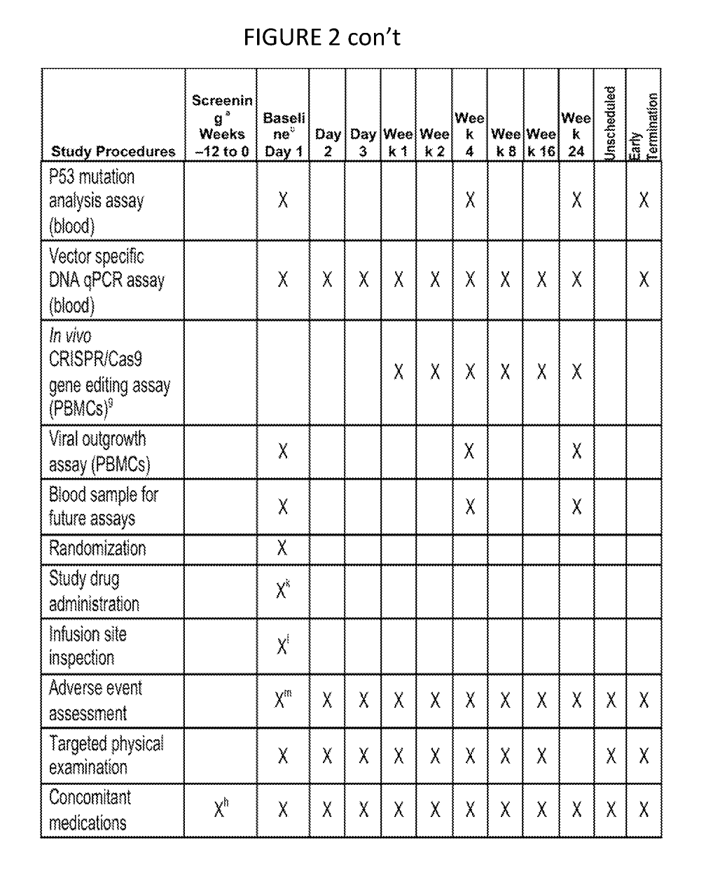

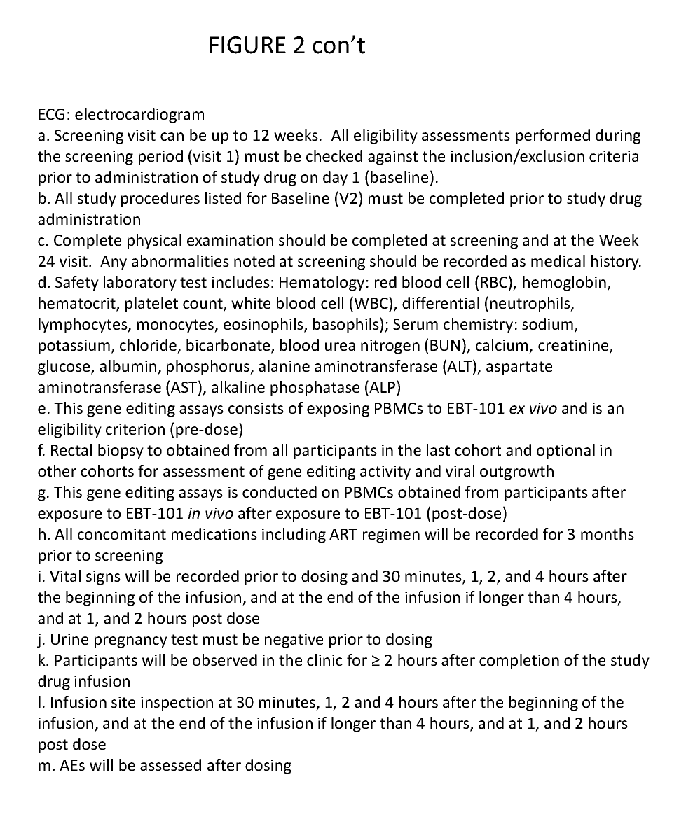

A schedule of study procedures performed in the method are summarized in FIG. 2. Computer software can be used for controlling, administrating, and reporting any aspects of the trial. The software can communicate with trial staff through smartphones, smart watches, tablets, and computers through wireless or wired signals to notify staff of timing of procedures, test results, or alerts with trial participants. The software can also send reminders to participants for clinic visits and follow ups.

On the first day of the study, the following study procedures are performed prior to treatment administration: targeted physical examination, vital signs, laboratory safety tests, CD4 T cell count, HIV-1 RNA (plasma), AAV9 antibodies (serum), urine pregnancy test, urinalysis, 12-Lead ECG, Rectal biopsy (optional), P53 mutation analysis, vector specific DNA qPCR assay (blood), viral outgrowth assay (PBMCs), blood sample for future assays, concomitant medications, and eligibility confirmation.

The treatment is administered to eligible participants who are observed in the clinic for ≥2 hours after completion of the study drug infusion. The following procedures are performed at 30 minutes, 1, 2, and 4 hours after the beginning of the infusion, at the end of the infusion if longer than 4 hours, and at 1 and 2 hours after the end of the infusion: infusion site inspection, vital signs, and adverse event assessment.

On the second day of the study, the following procedures are performed: adverse event assessment, vital signs, 12-lead ECG, vector specific DNA qPCR assay (blood), targeted physical exam, and concomitant medications.

On the third day of the study, the following procedures are performed: adverse event assessment, vital signs, 12-lead ECG, vector specific DNA qPCR assay (blood), targeted physical exam, and concomitant medications.

At the first week (approximately plus or minus a day), the following procedures are performed: adverse event assessment, vital signs, laboratory safety tests, CD4 T cell count, HIV-1 RNA (plasma), urinalysis, 12-Lead ECG, vector specific DNA qPCR assay (blood), In vivo CRISPR/Cas9 gene editing assay (PBMCs), targeted physical exam, and concomitant medications.

At the second week (approximately plus or minus a day), the following procedures are performed: adverse event assessment, vital signs, laboratory safety tests, CD4 T cell count, HIV-1 RNA (plasma), urinalysis, vector specific DNA qPCR assay (blood), In vivo CRISPR/Cas9 gene editing assay (PBMCs), targeted physical exam, and concomitant medications.

At the fourth week (approximately plus or minus a day), the following procedures are performed: adverse event assessment, vital signs, CD4 T cell count, HIV-1 RNA (plasma), AAV9 antibodies (serum), rectal biopsy (if obtained at baseline), p53 mutation analysis assay (blood), vector specific DNA qPCR assay (blood), In vivo CRISPR/Cas9 gene editing assay (PBMCs), viral outgrowth assay (PBMCs), targeted physical exam, and concomitant medications.

At the eighth week (approximately plus or minus two days), the following procedures are performed: adverse event assessment, vital signs, CD4 T cell count, HIV-1 RNA (plasma), vector specific DNA qPCR assay (blood), In vivo CRISPR/Cas9 gene editing assay (PBMCs), targeted physical exam, and concomitant medications.

At the 16^{th} week (approximately plus or minus two days), the following procedures are performed: adverse event assessment, vital signs, CD4 T cell count, HIV-1 RNA (plasma), vector specific DNA qPCR assay (blood), In vivo CRISPR/Cas9 gene editing assay (PBMCs), targeted physical exam, and concomitant medications.

At the final study visit (week 24, approximately plus or minus 2 days), the following procedures are performed: adverse event assessment, full physical examination, vital signs, laboratory safety tests, CD4 T cell count, HIV-1 RNA (plasma), AAV9 antibodies (serum), urinalysis, p53 mutation analysis assay (blood), vector specific DNA qPCR assay (blood), In vivo CRISPR/Cas9 gene editing assay (PBMCs), viral outgrowth assay (PBMCs), targeted physical exam, and concomitant medications. At the conclusion of the visit, participants are asked to sign a written informed consent form for the LTFU protocol.

Unscheduled visits can also occur if participants need additional follow-up for adverse events. The following study procedures can be performed as clinically indicated: adverse event assessment, vital signs, laboratory safety tests, CD4 T cell count, HIV-1 RNA (plasma), urinalysis, targeted physical examination, and concomitant medications.

If a participant prematurely discontinues the study for any reason following study drug administration, the investigator should make every attempt to bring the participant in for final clinic study visit and the following procedures can be performed as clinically indicated: adverse event assessment, vital signs, laboratory safety tests, CD4 T cell count, HIV-1 RNA (plasma), urinalysis, P53 mutation analysis, vector specific DNA qPCR assay (blood), targeted physical examination, and concomitant medications.

Phase 1a includes 6-18 participants treated with up to three different doses of the treatment (EBT101). Endpoints for this Phase 1a trial include (i) safety and tolerability and (ii) efficacy and accuracy of viral DNA excision as indexed by molecular biomarker analysis (PCR assays). The molecular assay provides information relating to the efficacy of the CRISPR therapeutic in a non-clinical phenotype-dependent manner. This efficacy data will be used to guide the design of the Phase 1b trial.

Phase 1b includes up to 32 participants treated in four equal cohorts: (i) single low dose, (ii) single high dose (0.5 to 1 log above low dose), (iii) two doses with a separation in time of 1-5 days, and (iv) placebo. As before, safety and tolerability are the primary endpoints and blood leukapheresis samples can be tested for the proper excision of the HIV genome using the PCR assay. An optimal dose can be determined by these trials. Phase 1c tests EBT101 in 24-32 participants in three cohorts: (1) 12-16 placebo, (2) 6-8 optimal dose (from Phase 1b) and (3) 6-8 at a 0.5 log higher dose than the Phase 1b optimal dose. As before, safety and tolerability are the primary endpoints. Blood leukapheresis samples can be tested for the proper excision of the HIV genome using the PCR assay.

In an analysis of the effectiveness of the gene editing or gene excising treatment, various assays can be performed to confirm HIV viral genome excision from the cells of the individuals in the trial. GI tract mucosal lymph node biopsies can be taken to test for HIV genome excision (molecular determination to be made in 1-2 days). Blood leukapheresis can be performed to test circulating T cells for HIV genome excision (molecular determination to be made in 1-2 days).

The assays as described above in Phases 1a-1c can include using a diagnostic panel to determine the effectiveness of the gene editing or excising treatment, as described in U.S. Provisional Patent Application No. 62/340,624. The diagnostic panel is able to detect biomarkers or metabolites indicative of the presence of a virus (HIV) that currently used PCR and ELISA assays in clinical trials are not able to detect. A sample can be taken from the individuals in the clinical trial at any point during the trial as necessary, the sample is applied to the diagnostic panel including at least one biomarker indicative of HIV, detecting the presence of at least one biomarker, comparing levels of the biomarker to a baseline, and determining if the treatment (EBT101) is working to reverse or prevent the HIV. The diagnostic panel can confirm that the HIV genome has been excised from the individuals' cells in the clinical trial. A baseline of healthy individuals can be chosen to compare to metabolite levels in individuals in the clinical trial. The metabolites can be measured in individuals having HIV in the clinical trial that are on HAART both before treatment begins and after treatment (at any point in the trial) to determine if the treatment is working.

The biomarkers are preferably metabolites that are indicative of the presence of a disease, and especially a virus (such as HIV). Metabolites are those chemicals (generally less than 1,000 Da) that are involved in cellular reactions for energy production, growth, development, signaling and reproduction, and can be taken up, or released from cells according to cellular needs. These chemicals include sugars, amino acids, organic acids, as well as xenobiotic compounds. Metabolomics (or metabonomics as it is sometimes referred), is dedicated to the study of all metabolites in a cell or system and changes that might result from an internal or external stress such as an infection, disease state, or exposure to a toxin. Metabolic changes can result from changes in the chemical reactions that use these metabolites (i.e. metabolic pathways), or the transporters that take up or release these metabolites. Infection of a person by a virus or bacterium causes major changes both at the cellular level (the site of infection), and systemically (through the innate immune response). These responses include, but are not limited to, signaling of specific immune cells, signaling of apoptosis, changes in transporters, as well as changes in mitochondrial function and energy production—changes that can be observed as changes in metabolite concentrations at the cellular level, and systemically in the blood or urine.

The metabolites can include, but are not limited to, 1,3-dimethylurate, levoglucosan, 1-methylnicotinamide, metabolite 1, 2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5 (which may be methylamine), metabolite 6 (which may be methylguanidine), N,N-dimethylglycine, O-acetylcarnitine, pantothenate, propylene glycol, pyroglutamate, pyruvate, quinolinate, serine, succinate, sucrose, metabolite 7 (which may be tartrate), taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, or glycerol.

The metabolite can also be any from the following metabolic cycles:

Polypurine: guanosine, guanine, xanthine, uric acid, adenosine, inosine, inosinic acid, hypoxanthine, xanthine, CO2, H2O, urea, N-carboamoyl-β-alanine, beta-alanine, ammonia, and (3-aminoisobutyrate.

Polyamines: putrescine, spermidine, spermine, methionine, S-adenosylmethionine, decarboxylated S-adenosylmethionine, arginine, ornithine, putrescine, N1-acetylspermidine, N1-acetylspermine, eIF5A(Lys), eIF5A(Dhp), eIF5A(Hpu), N1N2-diacetylspermine, 3-aminopropanal, 3-acetylaminopropanal, acrolein, and FDP-lysine protein.

KREBS/TCA cycle: threo-Ds-isocitrate, oxalo-succinate, 2-oxo-glutarate, oxalo-acetate, L-glutamate, 2-hydroxy-gluta rate, pyruvate, acetyl-CoA, cis-Aconitate, D-isocitrate, α-ketoglutarate, succinyl-CoA, succinate, fumarate, malate, glycine, citrate, carnitine, (−)O-acetyl-carnitine, cis-aconitate, itaconate, glycolate, glyoxylate, oxalate, oxalyl-CoA, formate, formyl-CoA, and CO₂.

Glycolysis and gluconeogenesis: glucose, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), pyruvate, D-glucose, D-glucono-1,5-lactone, D-gluconate, α-D-mannose 6-P, D-mannose, D-fructose, D-sorbitol, glycerone-P, sn-glycerol-3P, glycerol, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, and D-lactate.

Oxidative phosphorylation: adenosine triphosphate (ATP), adenosine diphosphate (ADP), H+, succinate, fumarate, H₂O, O₂, NADH, and NAD+.

Pentose phosphate: glucose-6-phosphate, NADP+, NADPH, 6-phosphogluconolatone, H₂O, H+, 6-phosphogluconate, CO₂, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, and xylulose 5-phosphate, D-ribulose, D-ribitol, D-ribose, L-ribulose, sedoheptulose 1,7P₂, 3-oxo-6-P-hexulose.

Urea cycle: L-ornithine, carbamoyl phosphate, L-citrulline, argininosuccinate, fumarate, L-arginine, urea, L-aspartate, adenosine diphosphate (ADP), adenosine monophosphate (AMP), and pyrophosphate.

Fatty acid β-oxidation: trans-A²-enoyl-CoA, L-β-hydroxyacyl CoA, β-ketoacyl CoA, FADH2, NADH, acetyl-CoA, acyl-CoA, propionyl-CoA, and succinyl-CoA.

Nucleotide metabolism: AMP, inosine monophosphate (IMP), xanthosine monophosphate (XMP), guanosine monophosphate (GMP), ribose-5-phosphate, adenosine, inosine, hypoxanthine, xanthosine, xanthine, guanosine, guanine, uric acid, fumarate, adenylosuccinate, uridine, uridine monophosphate (UMP), ADP, thymidine, thymine, deoxyribose-1-phosphate, deoxythymidine monophosphate (dTMP), deoxycytidine, ATP, and deoxycytidine monophosphate (dCMP).

Cofactors and vitamins: retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-C≡CH, NAD+, NADH, ADP, and ATP.

Amino acid metabolism: glutamate, NH4+, α-ketoglutarate, pyruvate, oxaloacetate, glutamate γ-semialdehyde, Δ¹-pyrroline-5-carboxylate, citrulline, arginine, urea, ornithine, glycine, CO2, NH3, N⁵,N¹⁰-methyleneTHF, 3-phosphoglycerate, α-ketobutyrate, propionyl-CoA, succinyl-CoA, acetyl-CoA, serine, α-amino-β-ketobutyrate, aminoacetone, cysteine sulfinate, β-sulfinylpyruvate, bisulfite, sulfite, sulfate, alanine, glutathione, taurine, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, tyrosine, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, carnitine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, quinolinate, glutaryl-CoA, and acetoacetyl-CoA.

A single metabolite can be used, as well as any combination of metabolites in determining disease state.

Various methods can be used to detect the presence of the biomarkers, such as, but not limited to, liquid chromatography, gas chromatography, liquid chromatography—mass spectrometry, gas chromatography—mass spectrometry, high performance liquid chromatography—mass spectrometry, capillary electrophoresis—mass spectrometry, nuclear magnetic resonance spectrometry (NMR), raman spectroscopy, or infrared spectroscopy.

Adverse Events

Adverse events (AEs) can be reported from study day 1 through the end of the study. It is the responsibility of the Investigator or Sub-Investigator(s) to perform periodic assessment of all AEs. Data describing AEs is entered in the participant's medical record and EDC, and as appropriate, a Serious Adverse Event (SAE) Report Form. SAEs can be reported to the Sponsor.

Causality assessment is required for AEs (and SAEs) that occur during clinical investigations. There is currently no standard international nomenclature to describe the degree of causality or relatedness of an AE with the study drug. The following terms are used during this study:

Likely—Reasons to consider an AE likely related to treatment include, but are not limited to the following: timing of the event relative to the administration of the investigational product, location of the AE relative to the site of investigational product administration, likelihood based on experience with similar products, there is a biologically plausible explanation based on the mechanism of action or mode of delivery of the treatment, the AE is repeated on subsequent treatments, and no other explanation is likely.

Unlikely—An AE with no temporal association with the study drug but rather related to other etiologies such as concomitant medications or conditions, or participant's known clinical state.

Severity can be reported in accordance with Common Terminology Criteria for Adverse Events (CTCAE) Version 5.0 Published: Nov. 27, 2017. If an appropriate listing is not present for an AE, the AE can be graded as follows:

Grade 1 (Mild)—No interference with daily activity.

Grade 2 (Moderate)—Some interference with daily activity but medical intervention not required (e.g., doctor visit and/or prescription medicine); over the counter medicine permitted.

Grade 3 (Severe)—Prevents daily activity and requires medical intervention (e.g., doctor visit and/or prescription medicine).

Grade 4 (Potentially Life-threatening)—Emergency room visit or hospitalization.

Any laboratory abnormality deemed clinically significant by the Investigator should be reported as an AE. A clinically significant abnormality is a confirmed abnormality (by repeat test) that is changed sufficiently from Screening/Baseline so that in the judgment of the Investigator a change in management is warranted. This alteration may include: monitoring the laboratory test further, initiating other diagnostic tests or procedures, changing ongoing treatment, or administering new treatment.

Whenever possible, the underlying medical diagnosis (e.g., anemia) should be reported as the SAE term. Repeated additional tests and/or other evaluations required to establish the significance and etiology of an abnormal result should be obtained when clinically indicated.

Any physical exam abnormality after study drug administration deemed clinically significant by the Investigator should be reported as an AE.

All pregnancies that occur including female partners of male participants during the study must be reported to the Sponsor and followed to conclusion. The outcome of each pregnancy must be reported. Pregnancy alone is not an AE, nor is an induced elective abortion to terminate a pregnancy without medical reason. However, an induced therapeutic abortion to terminate a pregnancy due to complications or medical reasons must be reported as an SAE. The underlying medical diagnosis for this procedure should be reported as the SAE term. A spontaneous abortion is always considered an SAE.

SAEs must be reported to the Sponsor or designee within 1 business day of becoming aware of the event. If the Investigator does not have all information regarding a serious adverse event, he/she will not wait to receive additional information before notifying the Sponsor (or designee) of the event and completing the SAE report. The SAE report will be updated when additional information is received. If at the time the Investigator submits an initial SAE report the event has not resolved, the Investigator must provide a follow-up report as soon as it resolves (or upon receipt of significant information if the event is still ongoing). All SAEs must be followed until resolution/stabilization or until a time that is mutually agreed upon between the Medical Monitor and the Investigator.

Upon checking “serious” on the AE eCRF, a notification will be sent to the Medical Monitor and/or designee. Relevant eCRFs, including the participant's Medical History, Concomitant Medications, and other AEs must also be completed to provide supporting documentation for the SAE. In addition, a SAE Narrative Form should be completed. After review of the initial SAE report, the Medical Monitor may request additional documentation (e.g., clinic or hospital records, or procedure reports).

The Sponsor is responsible for notifying the relevant regulatory authorities of certain events. It is the Investigator's responsibility to notify the Institutional Review Board (IRB) of all SAEs that occur at his or her site. Investigators will also be notified of all unexpected, serious, drug-related events that occur during the clinical trial. Each site is responsible for notifying its IRB of these additional SAEs.

Any participant experiencing an AE, including a clinically significant abnormal laboratory result or a physical exam finding, will be monitored at appropriate intervals (e.g., weekly for laboratory abnormalities) until resolution or stabilization, or until a time that is mutually agreed upon between the Medical Monitor and the Investigator.

Study Discontinuation

Participants who withdraw consent and/or refuse any further contact with respect to the study will be withdrawn from the study. Participants withdrawn from the study will not be replaced unless the withdrawal takes place prior to Week 8 visit post dose. All reasonable efforts will be made to prevent withdrawal of a participant from the study, including discussion of reasons for withdrawal and potential revised frequency of follow up visits that may be more acceptable to the participant. Telephone contact should be attempted at the very least.

Participants who sign and date the informed consent form but who fail to meet the inclusion and exclusion criteria are defined as screen failures. A screening log, which documents the patient's initials and reason(s) for screen failure, is to be maintained by the Investigator for all screen failures. A copy of the log should be retained in the Investigator's study files.

Conditions may arise during the study that could prompt the study to be halted or the study site to be terminated. Participants at such a site who have already received study drug will be transferred to another study site for follow up or another site will be established in the area to allow continued follow up of these participants.

Follow-Ups

A 15-day to 6-month (preferably 30-day) follow-up can be performed for safety and tolerability. A long term follow up (LTFU) protocol can be used to monitor participants for 15 years after completion of the study. All participants consenting to participate in the main protocol also can be asked to sign informed consent to participate in the LTFU Protocol. Participants can be reconsented when enrolled into the LFTU protocol. Long term monitoring includes annual clinical visits at the research site where the main protocol was conducted. In addition, health care workers (HCW) who provide care to the participants during the LTFU protocol can be given specific instructions regarding assessment and reporting of adverse events. Adverse events assessments can be ongoing during the LTFU period with special attention to: new malignancies, new incidence or exacerbation of pre-existing neurological disorders, new incidence or exacerbation of prior rheumatologic or other autoimmune disorders, and new incidence of hematologic disorders.

Statistical Analysis

This is a first-in-human study and no formal sample size calculations was conducted. The inclusion of 4 eligible participants per cohort (3 active and 1 placebo) and up to 5 cohorts (up to 20 total participants) is considered adequate to evaluate the preliminary safety, tolerability, immunogenicity and pharmacodynamics (PD) of EBT-101 and identify the likely maximum tolerated dose (MTD).

In general, descriptive statistics for continuous variables will consist of participant count, mean (or geometric mean), median, standard deviation (SD), and range. Descriptive statistics for categorical variables will consist of participant counts and percentages.

All other AEs will be coded using the Medical Dictionary for Regulatory Affairs (MedDRA) and summarized by cohort for frequency and severity. No formal statistical testing will be done. Clinically significant physical exam abnormalities after dosing will be reported as AEs, and summarized as described above.

Quantitative data (e.g. clinical lab results) will be summarized by mean, median, SD, and range. Laboratory abnormalities will be analyzed as safety outcomes by summarizing frequency, severity, and changes from baseline. Other analyses may include but are not limited to the following: examination of shift tables, pre-established severity grades, and change from baseline.

Serum anti-AAV9 antibody results will be analyzed descriptively by cohort and time post dose.

Plasma HIV-1 RNA will be be analyzed descriptively by individual participant, cohort and time post dose. Increases in viral load above 200 copies/mm3 will be examined closely in relation to other safety and PD endpoints.

The results of the vector specific DNA qPCR assays will be analyzed descriptively by cohort (dose), type of tissue (blood or other), and time post dose.

CRISPR/Cas9 HIV-1 gene editing activity with PBMCs and rectal lymph tissue will be analyzed.

Viral outgrowth in PBMCs and its correlation with gene editing activity will be analyzed descriptively by individual participant, cohort, and time post dose.

Concomitant medications can be coded using the most current World Health Organization (WHO) drug dictionary and summarized by drug class and medication term. Any changes in ART drugs or doses will be summarized separately.

Data Monitoring Committee

A formal independent safety review will be performed by an independent DMC according to a DMC charter established at the start of the study. After all participants from each cohort finish ≥2 weeks of post dose follow-up, the DMC will review available data.

The core DMC will consist of 3 members independent of the sponsor, including at least one physician with clinical expertise in gene therapy.

In general, the investigator(s) will not be involved in the closed data review process by the DMC. The investigator(s) will present relevant data in an open segment of the DMC review, and in the event of a suspected DLT or other clinically significant safety issue, the investigator may be invited by the DMC to participate in the open segment of the data review.

The DMC recommendations will include recommendations for opening the next cohort to enrollment. They also may include recommendations to continue the study as planned, continue the study with modification, or early termination of the study due to safety or administrative reasons. The working procedures of the DMC are described in a separate charter.

The DMC will be available to review the main study data through Week 24 of the last participant enrolled.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of performing a clinical trial for a gene editing or gene excising system for treating HIV in humans, including the steps of:
recruiting HIV infected individuals currently receiving highly active antiretroviral therapy (HAART) that is effective in lowering viral load and entering qualified individuals as participants in a clinical trial;
administering a gene editing or gene excising system treatment to the participants in Phase 1a, Phase 1b, and Phase 1c clinical trials; and
performing assays to confirm HIV viral genome excision from the participants' cells.

2. The method of claim 1, wherein the gene editing or gene excising system is a CRISPR system.

3. The method of claim 2, wherein the gene editing or gene excising system is EBT101.

4. The method of claim 3, wherein the EBT101 is formulated at a concentration of approximately 1×10³ vg/mL in phosphate buffered saline containing 180 mM sodium chloride, 10 mM sodium phosphate, and 0.001% Poloxamer 188.

5. The method of claim 2, wherein the CRISPR system includes a nuclease chosen from the group consisting of Cas9 and Cpf1.

6. The method of claim 1, wherein the gene editing or gene excising system is an Argonaute system.

7. The method of claim 1, wherein said recruiting step takes 3-12 weeks.

8. The method of claim 1, wherein the individuals receiving HAART that is effective in lowering viral load are determined by measuring no viral replication, no viral load of less than 20 copies/mm3, and healthy CD4 T cell counts of greater than 500/mm3.

9. The method of claim 1, wherein the HAART is chosen from the group consisting of nucleoside reverse transcriptase inhibitors, non-nucleoside reverse transcriptase inhibitors, protease inhibitors, fusion inhibitors, integrase inhibitors, CCR5 antagonists, and combinations thereof.

10. The method of claim 9, wherein the individuals remain on the HAART throughout the clinical trial.

11. The method of claim 1, wherein said recruiting step further includes the step of screening the individuals to assess eligibility for the clinical trial.

12. The method of claim 11, wherein the individuals must meet inclusion criteria of willing and able to provide written, signed informed consent prior to initiation of any study related procedures, age ≥18 years, documented HIV-1 infection, antiretroviral therapy (ART) for >1 year prior to screening, plasma HIV-1 RNA <200 copies/mL for ≥1 year prior to screening, stable ART for >3 months prior to screening and expectation that current ART regimen will be maintained throughout study duration, plasma HIV-1 RNA <20 copies/mL within 30 days prior to screening and during screening, peripheral blood CD4 T cell count >500 cells/mm3 within 30 days prior to screening and during screening, hemoglobin: ≥10.0 (males); ≥9.0 (females) g/dL, absolute neutrophil count (ANC): ≥1000/mm3, platelet count: ≥75,000/mm3, serum creatinine: ≥1.5 mg/dL (133 μmol/L), aspartate aminotransferase (AST) or alanine aminotransferase (ALT): ≤2.5 times the upper limit of normal (ULN), in blood sample from patient, ex vivo gene editing activity of EBT-101 demonstrated in at least 20% of transduced cells, willing and able to comply, as assessed by the investigator, with all study related procedures. willing and able to consent to enrollment in the LTFU protocol (15 years) after completing the main study, females of child-bearing potential must have a negative serum pregnancy test at screening and negative urine test prior to dosing, sexually active females of child-bearing potential must be willing to use one or more of the following forms of contraception from the time of providing written informed consent through the 24 week post dose visit: hormonal; barrier; intrauterine device (IUD); vasectomized partner; or abstinence, and sexually active males must be willing to use one or more of the above forms of birth control for either themselves or their partner, as appropriate, from the time of signing the informed consent form throughout the 24 week post dose visit.

13. The method of claim 12, wherein the individuals must not meet any of the exclusion criteria of use of any investigational product or investigational medical device within 30 days prior to screening, or anticipated use of any investigational agent prior to completion of all scheduled study visits through one year post dose, known or suspected hypersensitivity to any components of EBT-101 including formulation excipients and AAV9, pregnant or breastfeeding or planning to become pregnant or to breastfeed at any time through 24 weeks post dose, concurrent disease or condition that, in the opinion of the investigator, would interfere with study participation or safety, major surgery planned during the 24 week study period, history of malignancy with the exception of successfully treated basal cell or squamous cell carcinoma of the skin, history of HIV-related opportunistic infections within the past 5 years, receipt of any experimental HIV vaccine within the past year, receipt of any gene therapy product, approved or experimental, at any time, receipt of any licensed or experimental non-HIV vaccine within the 30 days prior to screening, acute or serious illness, in the opinion of the site investigator, requiring systemic treatment and/or hospitalization, within 30 days prior to screening, a history of organ transplantation or of chronic immunosuppressive therapy, receipt of any systemic immunomodulator within 60 days prior to screening or anticipated use during the 24 week study period, immune deficiency other than from HIV-1 infection, active drug or alcohol use or dependence that, in the opinion of the site investigator, would interfere with adherence to study requirements, and the presence of detectable anti-AAV9 antibodies.

14. The method of claim 3, wherein said administering step is further defined as administering EBT101 by intravenous infusion at a rate of greater or equal to 25 mL/hr at a volume of 100 mL by automatic volumetric infusion pump.

15. The method of claim 14, wherein said administering step is further defined as administering a dose of EBT101 chosen from the group consisting of a low dose of 1.0×10¹² vg/kg, a middle dose of 3×10¹² vg/kg, and a high dose of 1.0×10¹³ vg/kg.

16. The method of claim 1, wherein Phase 1a is further defined as including 6-18 participants treated with up to three different doses of the gene editing or gene excising system.

17. The method of claim 16, wherein said performing assays step is further defined as performing PCR assays and determining safety and tolerability and efficacy and accuracy of viral DNA excision.

18. The method of claim 1, wherein Phase 1b is further defined as including up to 32 individuals treated in four equal cohorts of single low dose, single high dose, two doses with a separation in time of 1-5 days, and placebo.

19. The method of claim 18, wherein said performing assays step is further defined as testing blood leukapheresis samples for proper excision of HIV genome with a PCR assay.

20. The method of claim 19, further including the step of determining an optimal dose of the gene editing or gene excising system.

21. The method of claim 1, wherein Phase 1c is further defined as including 24-32 participants in three cohorts of 12-16 participants in placebo, 6-8 in optimal dose, and 6-8 participants at a 0.5 log higher dose than the Phase 1b optimal dose.

22. The method of claim 1, wherein the Phase 1a, Phase 1b, and Phase 1c lasts 24 weeks for each participant.

23. The method of claim 1, further including the step of having clinic visits for safety endpoints at the baseline visit and on days 1, 2, and 3 post dose, and at 1, 2, 4, 8, 16, and 24 weeks post dose.

24. The method of claim 1, further including the step of obtaining blood samples from participants on baseline and at 1, 2, 4, 8, 16, and 24 weeks post dose.

25. The method of claim 1, further including the step of obtaining a rectal biopsy at baseline and 4 weeks for testing of gene editing activity.

26. The method of claim 1, wherein study procedures of targeted physical examination, vital signs, laboratory safety tests, CD4 T cell count, HIV-1 RNA (plasma), AAV9 antibodies (serum), urine pregnancy test, urinalysis, 12-Lead ECG, rectal biopsy, P53 mutation analysis, vector specific DNA qPCR assay (blood), viral outgrowth assay (PBMCs), blood sample for future assays, concomitant medications, and eligibility confirmation are performed on the first day prior to treatment.

27. The method of claim 19, wherein said performing assays step is further defined as testing blood leukapheresis samples for proper excision of HIV genome with a PCR assay.

28. The method of claim 19, wherein said performing assays step is further defined as confirming HIV viral genome excision from cells of the individuals by an test chosen from the group consisting of testing for HIV genome excision in GI tract mucosal lymph node biopsies and testing circulating T cells for HIV genome excision by blood leukapheresis.

29. The method of claim 1, wherein said performing assays step further includes using a diagnostic panel to determine the effectiveness of the gene editing or excising treatment that detects biomarkers or metabolites indicative of the presence of HIV.

30. The method of claim 29, wherein said using a diagnostic panel step is further defined as taking a sample from the individuals, applying the sample to the diagnostic panel including at least one biomarker indicative of HIV, detecting the presence of at least one biomarker, comparing levels of the biomarker to a baseline, and determining if the gene editing or gene excising system treatment is working to reverse or prevent the HIV.

31. The method of claim 29, wherein the biomarkers are chosen from the group consisting of 1,3-dimethylurate, levoglucosan, 1-methylnicotinamide, metabolite 1, 2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, eIF5A(Lys), eIF5A(Dhp), eIF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-C≡CH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, β-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

32. The method of claim 29, wherein the biomarkers are detected by a method chosen from the group consisting of liquid chromatography, gas chromatography, liquid chromatography—mass spectrometry, gas chromatography—mass spectrometry, high performance liquid chromatography—mass spectrometry, capillary electrophoresis—mass spectrometry, nuclear magnetic resonance spectrometry (NMR), raman spectroscopy, and infrared spectroscopy.

33. The method of claim 1, further including the step of reporting adverse events during the clinical trial.

34. The method of claim 1, further including the step of enrolling the participants in a long term follow-up protocol for annual clinic visits for 5 years post dose and telephone contact for 10 years for assessing adverse events.

35. A method of performing a clinical trial for a gene editing or gene excising system for treating a latent viral infection in humans, including the steps of:
recruiting infected individuals and entering qualified individuals as participants in a clinical trial;
administering a gene editing or gene excising system treatment to the participants in Phase 1a, Phase 1b, and Phase 1c clinical trials; and
performing assays to confirm viral genome excision from the participants' cells.

36. The method of claim 35, wherein the latent viral infection is chosen from the group consisting of hepatitis A, hepatitis B, hepatitis C, hepatitis D, HSV-1, HSV-2, coxsachievirus, cytomegalovirus, Epstein-Barr virus, Varicella Zoster virus, HIV1, HIV2, HSV-1, HSV-2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, varicella zoster virus, rota, seadornvirus, coltivirus, and BK virus.
